(19) United States
(12) Patent Application Publication
(10) Pub. No.: US 2024/0327245 A1
(43) Pub. Date: Oct. 3, 2024

(54) WATER PURIFICATION SYSTEM FOR SMART BUILDING

(71) Applicant: Geogrid Inc., Seongnam-si (KR)

(72) Inventor: Ki Hyun Kim, Namyangju-si (KR)

(73) Assignee: Geogrid Inc., Seongnam-si (KR)

(21) Appl. No.: 18/517,964

(22) Filed: Nov. 22, 2023

(30) Foreign Application Priority Data
Mar. 27, 2023 (KR) 10-2023-0039750

(51) Int. Cl. C02F 1/00; B01D 35/02; B01D 35/147

(52) U.S. Cl. CPC C02F 1/008; B01D 35/023; B01D 35/147; C02F 1/004; B01D 2201/54; B01D 2201/56; C02F 2209/006; C02F 2209/008; C02F 2301/043

(57) ABSTRACT

The present invention relates to a water purification system for a smart building that allows users to monitor a water pipe and a water quality condition and take disaster prevention response. The water purification system includes a water pipe scaler connected to the water pipe of the building and to scale an internal condition of the water pipe, a water pipe filtration device to filter a water supplied to the water pipe, a first sensor to measure a quality of the water flowing in the water pipe, a second sensor installed at the building or a ground of the building to measure a condition of the building or the ground, and a water supply control device to monitor the water quality and the condition of the building or the ground based on sensing data of the first sensor and the second sensor.

### CROSS-REFERENCE TO RELATED APPLICATIONS

This application claims priority to and the benefit of Korean (KR) Patent Application No. 10-2023-0039750, titled “Water Purification System for Smart Building,” filed Mar. 27, 2023, the entire disclosure of which is hereby incorporated herein by reference.

### FIELD OF THE INVENTION

The present invention relates to a water purification system for a smart building, and more specifically, to a water purification system for a smart building that allows users to monitor a water pipe and a water quality condition and take disaster prevention response using water quality information and building safety information collected through Internet of Things (IoT) devices installed in the water pipe of the building.

### BACKGROUND OF THE DISCLOSURE

With a development of technology, consumers' desire to drink clean water has led to the development of the water purifier industry, but overuse of plastic bottles containing spring water results in the production of a lot of electricity and the use of plastic, and results in a large amount of carbon emissions. According to Korea's general water supply system, Korea's water quality is so excellent that it ranks among the top 10 in the world, but an actual drinking rate of tap water is less than 5%. Rather than distrusting production and supply of clean tap water, consumers have a very high tendency to distrust that the water pipes in the building they live in are unclean, and the distrust rate is over 75%. This survey was made public as a survey conducted by the Korea Water Resources Corporation in 2016. Additionally, after this survey, the Korea Water Resources Corporation conducted an experiment showing that when water quality information was provided to a city in Gyeonggi-do, the drinking rate increased from 5% to 35%.

Accordingly, there is a need for measures to keep the water pipes clean, improve the water quality, and disclose the information to consumers to increase the drinking rate of tap water.

### PRIOR ART

Prior art related to this application includes: Korean Patent No. 10-1627793 (2016 May 31); Korean Patent No. 10-0676748 (2007 Jan. 25); Korean Patent No. 10-1183656 (2012 Sep. 11); Korean Patent No. 10-1585552 (2016 Jan. 8); Korean Patent No. 10-2101625 (2020 Apr. 10); Korean Patent No. 10-2440285 (2022 Aug. 31); and Korean Patent No. 10-2418300 (2022 Jul. 4).

### SUMMARY OF THE DISCLOSURE

This summary is provided to introduce a selection of concepts that are further described below in the detailed description. This summary is not intended to identify indispensable features of the claimed subject matter, nor is it intended for use as an aid in limiting the scope of the claimed subject matter.

The present invention provides a water purification system for a smart building that can allow users to monitor a condition of a water pipe and take disaster prevention response using water quality information and building safety information collected through Internet of Things (IoT) devices installed in the water pipe of the building.

In addition, the present invention provides a water purification system for a smart building that can improve satisfaction of all users by constructing a water pipe filtration device that is selectively used.

Technical problems to be achieved by the present invention are not limited to the technical problems mentioned above, and other technical problems not mentioned may be clearly understood by those skilled in the art from the description below.

According to an aspect of the present invention, a water purification system for a smart building may include: a water pipe scaler connected to a water pipe of the building and to scale an internal condition of the water pipe, a water pipe filtration device to filter a water supplied to the water pipe, a first sensor for measuring a water quality of the water flowing in the water pipe, a second sensor installed at the building or a ground of the building to measure a condition of the building or the ground, and a water supply control device to monitor the water quality and the condition of the building or the ground based on sensing data of the first sensor and the second sensor.

Furthermore, the water pipe filtration device may be disposed in a bypass pipe connected to the water pipe and selectively opened and closed.

Also, the water pipe filtration device may be composed of a plurality of devices, and may be disposed in each bypass pipe connected to the water pipe and selectively opened and closed.

In addition, the water supply control device may operate the water pipe scaler and the water pipe filtration device based on an analysis result of first sensing data of the first sensor.

Furthermore, the water supply control device may use a vibration sensor and a ground subsidence sensor as the second sensor, collect and analyze second sensing data of the second sensor, and perform a disaster prevention response operation based on an analysis result of the second sensing data.

Furthermore, the water supply control device may notify a condition of the water quality and the condition of the building or the ground to a user terminal owned by an owner of the building or a resident living in the building.

Also, the water supply control device may use an artificial intelligence (AI) program to analyze the first sensing data of the first sensor and public data acquired from public institutions related to the water quality, determine a water usage in a random area based on a result of the analysis, and ensure a water production according to a result of the determination.

The aspects of the present invention are only some of the preferred embodiments of the present invention, and various embodiments reflecting technical features of the present invention may be derived and understood by those skilled in the art by reference to detailed description of the present invention described below.

The present invention described above may include following effects.

The present invention may suppress and remove a creation of rust and foreign substances in a water pipe of a building, provide psychological stability and satisfaction to the building's residents by obtaining results from a water quality sensor and providing the results in real time, and consequently simultaneously increase value and make profit for business operators.

In addition, the national water office may determine an appropriate amount of water usage for households or regions by using water usage information, reduce energy used to overproduce by 15% throughout the country, and decrease carbon emissions.

In addition, in the case that a filtration device is applied to the water pipe that supply water to the building, the water quality may be improved and healthy water may be supplied by removing fine dust, pigments, microorganisms, chemicals, and etc., as a result, the quality of the water used inside the building may be improved, which can greatly contribute to the health and safety of users and increase user satisfaction.

In addition, when the water pipe filtration device is applied, impurities contained in the water may be reduced, which may reduce maintenance costs for the water pipe and other water-related facilities, increase lifespan of devices and facilities that handle water, and improve taste and odor of the water used inside the building.

Furthermore, the present invention may provide preemptive notification in the event of a problem and a process of supplying healthy water, so that consumers who are residents of the building may obtain reassurance and satisfaction that hygiene and health problems are solved through receiving information, and service providers may also include the benefit of providing various profit models.

The effects that may be obtained from the present invention are not limited to the effects mentioned above, and other effects not mentioned will be clearly understood by those skilled in the art from the description below.

These and additional aspects of the present disclosure are set forth in the description that follows, and/or may be learned by a person having ordinary skill in the art by reading the material herein and/or practicing the principles described herein. At least some aspects of the present disclosure may be achieved via means recited in the attached claims.

### BRIEF DESCRIPTION OF THE DRAWINGS

FIG. 1 is a diagram showing a water purification system for a smart building according to an embodiment of the present invention.

FIG. 2A is a diagram showing a simplified diagram of an Internet of Things device according to an embodiment of the present invention installed in a water meter.

FIG. 2B is a diagram illustrating how Internet of Things device according to an embodiment of the present invention is located within a water meter of an actual building.

FIG. 3A is a conceptual diagram showing a configuration of a water pipe filtration device according to an embodiment of the present invention.

FIG. 3B is a conceptual diagram showing a configuration of a water pipe filtration device according to an embodiment of the present invention.

FIG. 4 is a block diagram illustrating a detailed structure of the water supply control platform device of FIG. 1.

FIG. 5 is a flowchart showing a water supply-related platform service method according to an embodiment of the present invention.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for simplicity and clarity, and does not in itself dictate a relationship between the various embodiments and/or configurations discussed. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

Hereinafter, some embodiments of the present invention will be described in detail by illustrative drawings. In the case of adding reference numerals to components in each drawing, it should be noted that identical components are given the same reference numerals as much as possible even if the identical components are shown in different drawings. Additionally, when describing embodiments of the present invention, in the case that detailed descriptions of related known configurations or functions are judged to impede understanding of the embodiments of the present invention, detailed descriptions will be omitted.

Additionally, in describing components of embodiments of the present invention, terms such as first, second, A, B, (a), and (b) may be used. The mentioned terms are intended to distinguish the component from other components, and essence, sequence, or order of the component is not limited by the mentioned terms. In the case that a component is described as being “connected,” “coupled,” or “connected” to another component, the case may include a case that the component is directly connected or connected to another component, and a case that each component is “connected”, “coupled”, and “connected” through a third component.

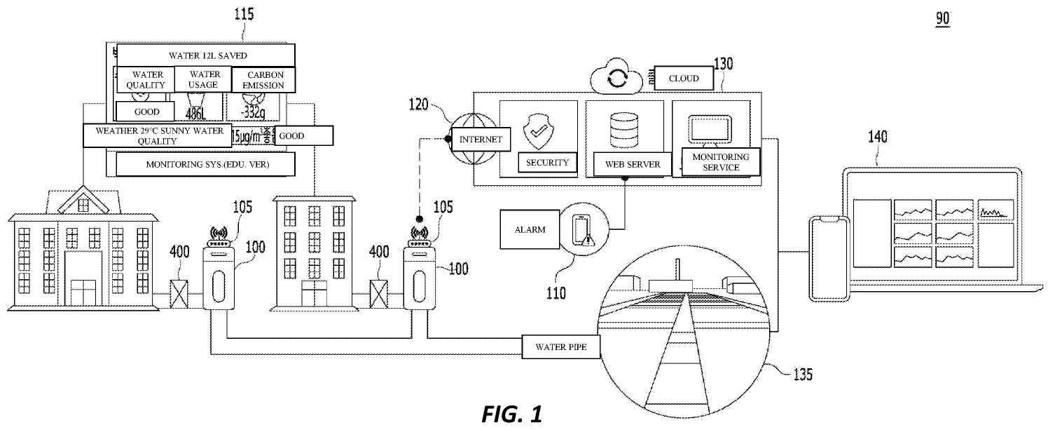

FIG. 1 is a diagram showing a water purification system for a smart building according to an embodiment of the present invention. As shown in FIG. 1, the water purification system for the smart building 90 according to an embodiment of the present invention may include part or all of a water supply equipment 100, 105 (e.g., an Internet of Things device for water supply, not shown), a user terminal 110, a home pad 115, a communication network 120, a water supply control device 130, a water supply office device 135, an administrator device 140, and a water pipe filtration device 400.

A disclosure of “including part or all” disclosed above may include a case that the water purification system for the smart building 90 is configured by omitting some components such as the home pad 115 or the administrator device 140, and a case that some or all of the components that make up the water supply control device 130 are integrated into a network device (e.g., wireless exchange device) that makes up the communication network 120, however, following will be described in full to facilitate a sufficient understanding of the present invention.

The water supply equipment 100, 105 may include various types of sensors configured inside the water meter 100 and a water pipe scaler that is connected to a water pipe and scales the water pipe. The water pipe scaler may also be called ionizer. The water pipe scaler may use carbon in tap water to generate static electricity to improve a water quality. By installing the water pipe scaler at the water pipe, rust, scale, slime (e.g., scale), and general bacteria within the water pipe may be removed. The water supply equipment 100, 105 may include a communication terminal 105 for transmitting data processed by various devices configured inside the water meter 100 to the communication network 120 and providing the data to the water supply control device 130. The communication terminal 105 may include a wired communication method, but the communication terminal 105 may be preferable to provide the data to the water supply control device 130 through a wireless communication method, such as a wireless router installed at home. The communication terminal 105 may be manufactured and used separately for operation according to an embodiment of the present invention.

The water supply equipment 100, 105 may include a first sensor such as a water quality sensor provided inside the water meter 100 for sensing the water quality of water flowing inside the water pipe. In addition, the water quality sensor may be installed in various forms in the water pipe to sense the water quality. For example, the water quality sensor according to the embodiment of the present invention may be installed in up to three places in the water pipe, a first sensor set may be installed in the water pipe at starting point where the water flows in, the ionizer (or water pipe scaler) may be installed at the water pipe behind the first sensor set, a second sensor set may be installed in the water pipe behind the ionizer, and a third sensor set may be installed at the very end of the water pipe. Above configuration may improve accuracy of water quality measurement. The water quality sensor may consist of a set of various water quality sensors for each function, and may also include a vibration sensor. The vibration sensor may measure vibration of the water pipe or the buildings where the water pipe is installed. The water quality sensor may operate by receiving electricity at all times and may transmit data information, that is, sensing data, in real time or on a minute basis to the water supply control device 130 through the Internet network. The water quality sensor may measure water temperature, conductivity, total dissolved solids (TDS), turbidity, salinity, oxidation reduction potential (ORP), and etc.

Additionally, the water supply equipment 100, 105 may include a second sensor installed inside the water meter 100 or the building for sensing safety of the building. The vibration sensor of the second sensor may be configured together with the water quality sensor, but in an embodiment of the present invention, the vibration sensor or ground a sensor may be installed in the building or the ground around the building to provide building safety information and to be implemented disaster prevention response action based on the sensing data collected through the vibration sensor. The second sensor, like the first sensor, may provide the sensing data to the water supply control device 130 in real time or on a per-minute basis to be stored in a cloud format. In addition, the second sensor according to an embodiment of the present invention may further include various sensors capable of measuring a tilt of the building or cracks of the building. An angle sensor or a tilt measurement sensor may be used to measure the tilt. Purpose of measuring the vibration of the building through the second sensor may include a purpose of disaster prevention response and a purpose of determining or predicting changes in the condition of the water pipe by measuring mutual relationship with the water pipe.

Furthermore, the water pipe scaler, that is, the ionizer, may be provided inside the water meter 100. More precisely, the water pipe scaler may be located at lower part of the water meter 100 with water pipe connected to both sides of the water pipe scaler. The water pipe scaler may be remotely controlled from the water supply control device 130 of FIG. 1. In other words, in the case that the water quality shows abnormal values as a result of water quality measurement, various operations may be performed to bring the water quality to normal values. Typically, the water pipe scaler may sterilize general bacteria, remove and suppress pipe rust, suppress and remove scale adhesion, and remove and suppress slime (e.g., scale). Since the water pipe scaler may be configured in various forms to perform the above operations, embodiments of the present invention will not be specifically limited to any one form. For example, when tap water passes through internal passage of the water pipe scaler, the water pipe scaler may spray a harmless disinfectant for sterilization into the water. Above all, the water pipe scaler according to an embodiment of the present invention may purify the water by generating static electricity using carbon in tap water. Therefore, by installing the water pipe scaler, rust, scale, slime, and general bacteria within the water pipe may be removed. The scale may refer to film-like impurities and metal oxides attached to metal surface, and may also be referred to as a capstone.

Additionally, a system for preventing freezing may be configured inside the water meter 100. The freezing prevention system may include a heat exchanger and a temperature sensor. In addition, operation of the heat exchanger or temperature sensing data by the temperature sensor may be provided to the communication terminal 105 so that the water supply control device 130 may control the heat exchanger and the temperature sensor. For example, in the case that there is a possibility of freezing and bursting of the water meter 100 as a result of temperature sensing of the temperature sensor, the heat exchanger may be operated by remote control and the operation of the heat exchanger may be stopped when internal temperature of the water meter 100 reaches set temperature. In addition, the heat exchanger may operate without a separate remote control depending on an operation of the temperature sensor, but the operation data or temperature sensing data of the heat exchanger may be transmitted to the water supply control device 130 of FIG. 1 for monitoring.

In addition, the water purified by the water pipe scaler may be filtered again by the water pipe filtration device 400. The water pipe filtration device 400 may perform a multi-step filtering process to remove various impurities contained in the water supplied into the building mainly through the water pipes. For example, following filtering process may be performed. The processes such as coagulation, flocculation, sedimentation, and filtration may be included. The ‘flocculation’ is a process of adding a coagulant to collect impurities to form impurities into clusters, the ‘flocculation’ is a process of slowly stirring the coagulated impurities to form lumps, the ‘sedimentation’ is a process of impurities settling and floating on water surface, and the ‘filtration’ is a process of removing impurities through a filter before precipitated impurities float on the water surface. Through these processes, impurities, homogeneous substances, chemicals, etc. contained in the water may be removed to maintain the high water quality.

The water pipe filtration device 400 may be applied to the building in various industrial fields. For example, the water pipe filtration device 400 may be used to improve the water quality and productivity by treating large amounts of water in semiconductor, electronics, pharmaceutical, food, beverage, and chemical industries. In detail, the water pipe filtration device 400 may be used in a manufacturing process of a manufacturing plant to improve the water quality and thus increase quality and safety of products, in the food and beverage industry to improve taste and odor and remove microorganisms and chemicals, and in the hospitals to clean medical equipment or purify the water used at patient beds since clean and safe water is needed.

In order to be used in various fields, the water pipe filtration device 400 may include various filters. For example, a filtration membrane filter, an activated carbon filter, an ion exchanger, an injection filter, an inverted triangle filter, a magnetic filter, an oxygen coefficient filter, and a gravity filter may be applied to the water pipe filtration device 400.

The filtration membrane filter may be used to remove fine particles and impurities. The filtration membrane filter may be composed of fibrous membranes that capture fine particles while allowing the water to flow, and generally be used in reverse osmosis (RO) and ultrafiltration (UF) systems.

The activated carbon filter may be used to remove odors and tastes. The activated carbon filter may be made up of activated carbon, and capture impurities and chemicals while allowing the water to flow.

The ion exchanger may be used to remove or exchange ions from the water, and generally be used to remove ions such as chlorine, sulfur, and nitrogen.

The injection filter may be a method of filtering water to be filtered by spraying a nutrient solution, when the water flows inside the filter under pressure, chemicals for filtering may be injected together with the water. The chemicals may remove impurities and contaminants in the water.

The inverted triangle filter may be installed in a purified water tank, and after the water flows to bottom of the tank, impurities and contaminants may be filtered out as the water passes through filtering medium and rises to top of the tank.

The magnetic filter may be a filter that uses a magnet to remove iron-based substances such as iron powder or rust from the water, and due to magnetic field generated by the magnet, the substances in the water may stick to the magnet and be filtered.

The oxygen coefficient filter may be a filter that removes bacteria and viruses in the water by adding oxygen to the water, supply oxygen to the water through an oxygen supply device, and remove bacteria and viruses in the water by using the supplied oxygen.

The gravity filter may be a filter in which the water is filtered by gravity of the water and filtering medium inside the filter, the filtering medium may be fixed, and impurities and contaminants may be filtered out as the water flows down from top of the filtering medium.

Meanwhile, when using the water pipe scaler and the water pipe filtration device 400 together, the water pipe scaler may generally be installed first and then the water pipe filtration device 400 may be installed. In the case of installing the water pipe scaler first, the water pipe scaler may serve to remove scale inside the water pipe, and the water pipe filtration device 400 may serve to purify the water, so the internal scale of the water pipe may be reduced and filtering operation of the water pipe filtering operation 400 may be performed more efficiently. In addition, a reverse installation may also be possible.

At least one first sensor for measuring the water quality of the water flowing in the water pipe may be disposed to measure the water quality before and after using the water pipe filtration device 400.

Operation data of the water pipe filtration device 400 or the sensing data through the first sensor may be transmitted to the water supply control device 130 of FIG. 1 for monitoring.

In addition, the water pipe filtration device 400 may also be remotely controlled and operated by the water supply control device 130 of FIG. 1. In other words, in the case that the water quality shows abnormal values as a result of the water quality measurement, the water pipe filtration device 400 may perform various operations to bring the water quality to the normal values.

The user terminal 110 may refer to a terminal owned by the building owner or the residents of the building where the water pipes are installed. The user terminal 110 may be preferably a mobile-based terminal, such as a smartphone or a wearable device that users wear on users' wrists. The building owner or the residents of the building who possess the user terminal 110 may receive information related to the water quality of the water they are currently using or condition of the building from the water supply control device 130. To use these services, the building owner or the residents may install an application (hereinafter referred to as an App) and receive the information in the form of a push message. Additionally, status notifications may be received in the form of SMS text messages, email, or messenger-based channels that enable chat. Above all, the information provided from the water supply control device 130 to the user terminal 110 may be provided through an online subscription-type paid service.

The home pad 115 may refer to a pad device installed inside the building, such as a detached house, an apartment, or an office. Typically, when a call button of a household call device installed at a door outside the building is pressed, the home pad 115 may be used to activate a camera and to check a person inside the building who pressed the call button. However, in the embodiment of the present invention, a program may be installed on the home pad 115 to perform operations according to the embodiment of the present invention, so that the building owner, etc. may check the water quality or the building condition through the home pad 115. Additionally, the operation above mentioned may be performed under a control of the water supply control device 130 of FIG. 1, and in the case of an emergency, a pop-up window notifying crisis may be displayed on a screen to inform the water quality information or the dangerous condition of the building.

The communication network 120 may include both wired and wireless communication networks. Additionally, a wired or wireless Internet network may be used or linked as the communication network 120. The wired network may include the Internet network such as a cable network or public switched telephone network (PSTN), and the wireless network may include code division multiple access (CDMA), wideband code division multiple access (WCDMA), global system/standard for mobile communication (GSM), evolved packet core (EPC), long term evolution (LTE), wireless broadband internet (Wibro), and etc. However, the communication network 120 according to an embodiment of the present invention is not limited to above mentioned network, and may include an access network of a next-generation mobile communication system to be implemented in the future, such as a cloud computing network in a cloud computing environment and a 5G network. For example, in the case that the communication network 120 is the wired communication network, an access point within the communication network may be connected to a telephone company's exchange office, and in the case that the communication network 120 is the wireless communication network, the access point may be connected to serving GPRS support node (SGSN) or gateway GPRS support node (GGSN) operated by communication company to process data, or connected to various repeaters such as base transceiver station (BTS), NodeB, and e-NodeB to process data.

The communication network 120 may include the access point. The access point may include small base stations such as femto or pico base stations that are often installed in the building. The femto or pico base stations may be classified according to a maximum number of the communication terminals 105, the user terminal 110, the home pad 115, and etc. that may be connected according to classification of small base stations. In addition, the access point may include a short-distance communication module for performing short-range communication, such as ZigBee or Wi-Fi, with the communication terminal 105, the user terminal 110, and the home pad 115. The access point may use transmission control protocol/internet protocol (TCP/IP) or real-time streaming protocol (RTSP) for the wireless communication. In addition, the short-range communication may include various standards such as Bluetooth, Zigbee, infrared (IrDA), radio frequency (RF) such as Ultra High Frequency (UHF) and Very High Frequency (VHF), and ultra-wideband communication (UWB) in addition to the Wi-Fi. The access point may extract location of data packet, designate the best communication path for the extracted location, and forward the data packet to next device, such as the water supply control device 130, along the designated communication path. The access point may share multiple lines in a typical network environment and include a router and a repeater.

The water supply control device 130 may provide a smart Internet of Things (IoT) water supply control platform service according to an embodiment of the present invention. Term of platform may refer to an online service, and also refer to an environment built so that multiple groups, such as platform suppliers and consumers, may participate and exchange value that each group wants to obtain through fair transactions. A dictionary meaning of the platform may refer to a specific processor model that is basis of the system and an operating system based on one computer system, for example, the platform may mean an operating program within a server. A service provider may install products, equipment, or facilities for operation according to embodiments of the present invention in the users' water pipe or the building, monitor, provide the water quality and the building safety information to users through the Internet, and regularly receive compensation for a certain fee in return for services. In other words, a monthly fee may be charged to service users.

The service users may achieve an effect of suppressing water pipe corrosion through products or equipment installed in the building's water pipe, such as water supply equipment 100, 105, may obtain the water quality information from the water quality sensors collected in real time, and may benefit from disaster prevention response by using the building safety information from the vibration and ground sensors, and the service provider may also perform operations such as charging and settling fees to the service users to provide various profit models. For example, the service users may be divided into general user and corporate user who may receive detailed raw data, and unlike the general user, the corporate user may be able to provide other additional services by utilizing the detailed raw data.

The water supply control device 130 may collect the water quality data through the water quality control platform, more precisely, the Internet of Things devices, which are water supply equipment 100, 105 where the water meter 100 is installed. The water quality data may include the water temperature, conductivity, TDS, turbidity, salinity, and ORP information. Additionally, the water supply control device 130 may improve the water quality and perform preventive operations. In other words, the preventive operations such as regular water quality improvement, greening prevention, water softening, and foreign substance formation suppression may be performed through the water pipe scaler and water pipe filtration device 400, which are installed at the lower part of the water meter 100 and connected to the water pipe. To this end, the water supply control device 130 constantly may monitor the water pipe and the water quality condition, and when an abnormal reaction is observed or the water quality condition deteriorates below set-up standard, the abnormal reactions and the water quality may be improved by operating the water pipe scaler and/or the water pipe filtration device 400.

Furthermore, the water supply control device 130 may utilize data information from public institutions to analyze the collected sensing data and provide the results to the service users. For example, the water supply control device 130 may predict water usage on a daily, monthly, and yearly basis by using the water quality information for the consumer building, weather information (e.g., rainfall, precipitation, atmosphere, ozone, fine dust, temperature, and etc.) provided by the public institutions, and other public information (e.g., traffic, electricity, gas, and etc.), also predict the changes in the water quality, and also predict the water quality and the water usage, such as leakage prediction and regional use prediction, in a specific period of the future. For the predictions above mentioned, the water supply control device 130 may build an artificial intelligence data system, in other words, the water supply control device 130 may produce an appropriate amount of the water at the national water supply office by using an artificial intelligence deep learning program. In this way, by predicting water usage and knowing the appropriate amount of water usage, an appropriate amount of tap water may be produced, thereby reducing energy used for overproduction by 15% throughout the country and reducing carbon emissions.

In addition, the water supply control device 130 may predict condition changes due to correlation between the vibration of the building and the water pipe by linking the water quality sensor, and the vibration detection sensor and the ground subsidence detection sensor to diagnose the safety of the building, and provide an additional building safety to the service users by converting the tilt and crack sensor of the building into data. The users may receive the water quality information and diagnostic information in real time and additional future forecast information, which allows the service providers to generate profits through purchase costs for product installation and provision of additional information.

In addition, the water supply control device 130 may control operating conditions of the water pipe filtration device 400, that is, control filtering time, flow, pressure, pH, concentration, and etc.

The water supply office device 135 may include various devices such as a computer installed and operated at a water supply office that manages water supply in a specific area. The water supply office device 135 may provide public data to the water supply control device 130 according to an embodiment of the present invention. However, in the embodiment of the present invention, there will be no particular limitation to receiving public data from the water supply office. The public data may include data such as Geographic Information System (GIS) information, weather information, fine dust, precipitation, and carbon emissions. In addition, the water supply office device 135 may include a fully automatic valve connected to the water pipes. The fully automatic valve may also be controlled by the water supply office device 135. Above all, the water supply office device 135 may receive predicted information on water production provided by the water supply control device 130 of FIG. 1 and adjust the water production based on the predicted information.

The administrator device 140 may include devices such as a computer, smartphone, tablet PC, etc. owned by an administrator who manages service of the water supply control device 130 of FIG. 1 or an administrator of the water supply office. Additionally, the administrator device 140 may include a status board in a form of a display installed in a control center that controls the water supply. For example, the water supply control device 130 may provide an optimal solution to the water supply office device 135 by analysis using water usage data and public data, and also perform a forecasting operation (e.g., information collection of the Korea Meteorological Administration and Water Resources Corporation). Through the forecasting operation, sudden situations may be prepared in advance.

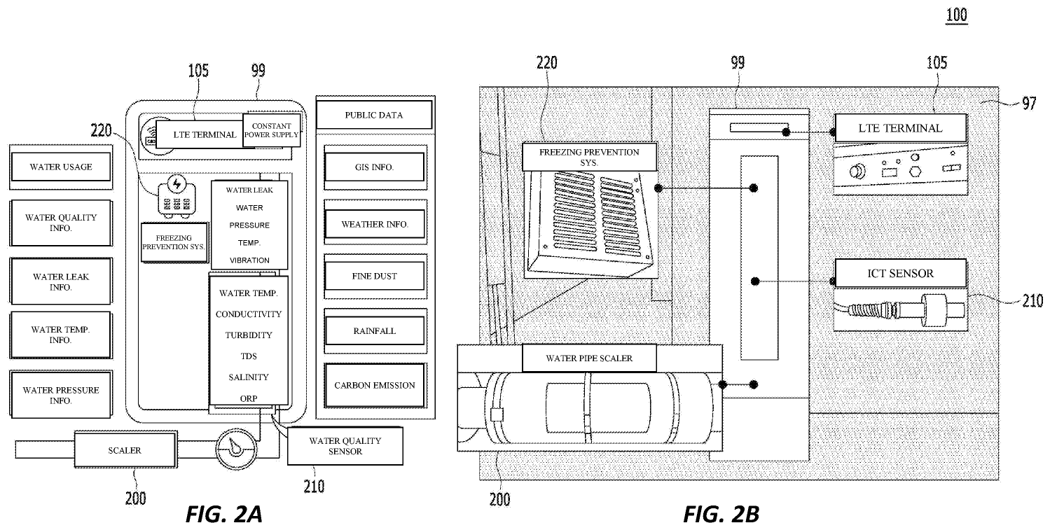

FIG. 2A is a diagram showing a simplified diagram of an Internet of Things device according to an embodiment of the present invention installed in a water meter and FIG. 2B is a diagram illustrating how Internet of Things device according to an embodiment of the present invention is located within a water meter of an actual building.

As shown in FIG. 2, the water supply equipment 100, 105 according to an embodiment of the present invention may include part or all of configurations installed inside the water meter 100, which may include a water pipe scaler 200, a sensor 210, a freezing prevention system 220, and the communication terminal 105.

A description “including part or all” above disclosed may include a case that the water supply equipment 100, 105 is configured by omitting some components such as the freezing prevention system 220, and a case that a component such as the sensor 210 is integrated with another component such as the water pipe scaler 200, however, following will be described in full to facilitate a sufficient understanding of the present invention.

The water pipe scaler 200 may be located at a bottom of the main body 99 of the water meter 100. A meter for water quantity reading may be installed at a lower part of the water meter 100, and the water pipe scaler 200 may be installed before the meter based on direction in which the water is supplied. The water quality sensor may be installed on one side and the other side of the water pipe scaler 200. In addition, the water pipe scaler 200 may be remotely controlled and operated by the water supply control device 130 of FIG. 1. Additionally, the communication terminal 105 may control the water pipe scaler 200 at the request of the water supply control device 130. For example, when the water quality shows the abnormal values or it is determined that an inside of the water pipe needs to be cleaned, a corresponding action may be performed by the remote control. Through this, the quality of tap water may be improved. The results of the improved water quality may be sensed through the water quality sensor and provided to the water supply control device 130 of FIG. 1 for monitoring, and the water quality improvement information may be provided to the building owner or the residents of the building. The water pipe scaler 200 according to an embodiment of the present invention may improve the water quality by generating static electricity using carbon in tap water.

The sensor 210 may include a first sensor and a second sensor. The first sensor may be installed in the water pipe and include the water quality sensor that senses mercury, conductivity, turbidity, TDS, salinity, ORP, etc. In addition, the second sensor may include the vibration sensor installed in the building 97 surrounding the water pipe or water meter 100, the ground sensor installed in the ground to sense whether the ground is subsided, the tilt sensor that measures tilt, or the crack sensor that measures crack of the building 97. The sensing data sensed by these various types of sensors may be provided to the water supply control device 130 of FIG. 1 through the communication terminal 105.

The freezing prevention system 220 may include the heat exchanger and may further include the temperature sensor. The freezing prevention system 220 may be located at an upper side inside the main body 99 of the water meter 100. However, the communication terminal 105 may be configured to be mounted at an outer upper side of the main body 99 of the water meter 100. The temperature sensor may measure an internal temperature of the water meter 100 and provide a measured value to the communication terminal 105 so that the water supply control device 130 may monitor the internal temperature of the water meter 100. The water supply control device 130 may control the heat exchanger according to monitoring results and stop the control when set temperature is reached. In addition, the heat exchanger may be automatically operated according to the temperature measured through the temperature sensor, and operation data according to the operation may be provided to the water supply control device 130. According to the operation data, the water supply control device 130 may monitor whether the freezing prevention system 220 is operating normally. In order to generate big data, the water supply control device 130 may periodically collect the operation data and the temperature data of the freezing prevention system 220, systematically classify and store the operation data and the temperature data at a web server of FIG. 1, and then may perform big data analysis. The freezing prevention system 220 may participate in the operations introduced above.

Also, as seen in FIG. 2, the communication terminal 105 may be provided on the main body 99 constituting the water meter 100. In addition, when the communication terminal 105 is exposed to the outside, the communication terminal 105 may include a communication box to protect a communication device from surrounding environment such as external shock, rain, and wind, and the communication terminal 105 may be installed inside the communication box. In the embodiment of the present invention, an LTE terminal may be exemplified, but since communication may be performed using various communication standards, the communication terminal 105 according to the embodiment of the present invention will not be limited to any one specific type.

In addition to the above disclosure, the water pipe scaler 200, the sensor 210, the freezing prevention system 220, and the communication terminal 105 installed inside the water meter 100 may perform various operations, but since the details are previously disclosed, further description is omitted.

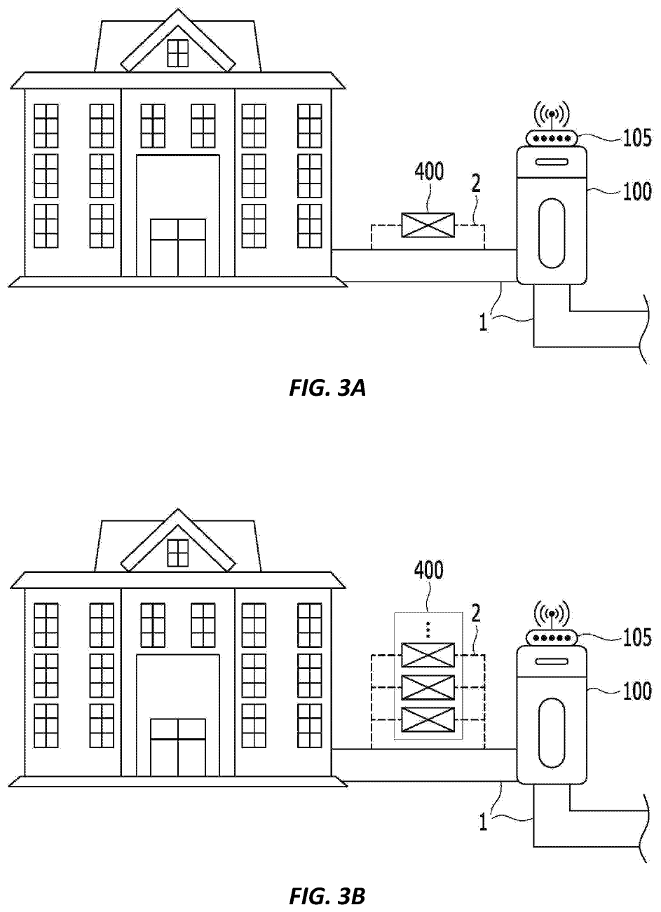

FIG. 3 is a conceptual diagram showing a configuration of a water pipe filtration device according to an embodiment of the present invention. As shown in FIG. 3A, a water pipe filtration device 400 may be installed at a water pipe 1 leading to the building (reference no. not shown), and the water pipe filtration device 400 may be disposed between the water meter 100 including the water pipe scaler (not shown) and the building to which the water pipe 1 is connected. Additionally, the water pipe filtration device 400 may include a bypass pipe 2 that is connected to the water pipe 1 and is selectively opened and closed.

To the building, the water may be supplied through the water pipe 1 after passing through only the water pipe scaler, or the water may be supplied through the water pipe 1 after passing through both the water pipe scaler and the water pipe filtration device 400. For two cases of water supply, the water pipe 1 and the bypass pipe 2 shown in FIG. 3 may include at least one open/close valve (not shown). The open/close valve may be composed of an electronic valve controlled by a water supply control device. In addition, in the drawing, the bypass pipe 2 may be briefly depicted as a single dotted line to identify the water pipe 1 and the bypass pipe 2.

Meanwhile, as described above, the water pipe filtration device 400 may be comprised of a plurality of devices. Therefore, as shown in FIG. 3B, it may be composed of a plurality of water pipe filtration devices 400, and filtered water may be supplied through only the desired water pipe filtration device 400 according to the user's request. Since the user may select some of the plurality of water pipe filtration devices 400, therefore, the bypass pipe 2 may be selectively opened and closed according to the water pipe filtration devices 400 selected by the user.

Additionally, the user and/or the office may pay a monthly usage fee depending on a number of the water pipe filtration devices 400 used and a type of the water pipe filtration device 400. For example, assuming a single building with the water pipe filtration device 400 as a filter, a hospital located on the 2nd floor may use the aforementioned filtration membrane filter, activated carbon filter, ion exchanger, and oxygen coefficient filter, and a general office on the 3rd floor may only use the oxygen coefficient filter. In addition, offices of the same industry may be placed on a specific floor, and the water using specific filter may be supplied to the specific floor. In addition, at least one filter may be configured in one bypass pipe 2, and a plurality of bypass pipes 2 may be configured and at least one filter may be configured for each bypass pipe 2.

FIG. 3 is a block diagram illustrating a detailed structure of the water supply control device of FIG. 1. As shown in FIG. 3, the water supply control device 130 of FIG. 1 according to an embodiment of the present invention may include part or all of a communication interface 300, a controller 310, a water supply control unit 320, and a storage 330.

A description of “including part or all” disclosed above may include a case that the water supply control device 130 is configured by omitting some components such as the storage 330, and a case that some components, such as the water supply control unit 320, are integrated into other components such as the controller 310, however, following will be described in full to facilitate a sufficient understanding of the present invention.

The communication interface 300 may communicate with each of the communication terminal 105, the user terminal 110, the home pad 115, the water supply office device 135, and the administrator device 140 included in the water supply equipment 100, 105 of FIG. 1. The communication interface 300 may perform operations such as modulation/demodulation, muxing/demuxing, encoding/decoding, and scaling to convert resolution during communication process, but since the above-mentioned operations are obvious to those skilled in the art, no further explanation is required.

The communication interface 300 may receive the sensing data from various sensors provided inside or near the water meter 100. More precisely, the sensing data may be received from the water supply equipment 100, 105 installed in the water meter 100. The sensing data may include not only data related to the water quality measurement, but also sensing data such as the vibration condition of the building or the subsidence condition of the ground. The communication interface 300 may receive the data and transmit the data to the controller 310.

Additionally, the communication interface 300 may perform various operations under control of the controller 310 according to analysis results of the sensing data. Typically, when the water quality needs to be improved, the communication interface 300 may control the operation of the water pipe scaler 200 included in the water meter 100 by communicating with the communication terminal 105 of FIG. 1. In addition, a corresponding operation may be performed by the communication terminal 105 as a control device that controls the water pipe scaler 200 according to a remote control of the water supply control device 130 of FIG. 1, that is, according to a control command. Accordingly, the water pipe scaler 200 may become a controlled device of the communication terminal 105.

Furthermore, the communication interface 300 may provide information related to the water quality or the building condition to the user terminal 110 or the home pad 115 under the control of the controller 310. For example, in the case that an earthquake occurs, the building may shake, which may cause changes in the condition of the water pipes. The user terminal 110 or the home pad 115 may receive such the information, and the communication interface 300 may be involved in the operation of transmitting such the information.

In addition, the communication interface 300 may communicate with the water supply office device 135 to provide relevant information to control the water production. In other words, in the case that the water supply control device 130 of FIG. 1 predicts the amount of tap water used at a specific water supply office and determines that water production volume needs to be adjusted, the communication interface 300 may transmit corresponding information. The communication interface 300 may perform a type of forecast for controlling the water production. In addition, the communication interface 300 may communicate with the administrator device 140 to provide various types of data, for example, in the case that the administrator device 140 may access service of the water supply control device 130 to check data such as forecast statistics, the communication interface 300 may provide the corresponding data.

The controller 310 may perform overall control operation of the communication interface 300, the water supply control unit 320, and the storage 330 of FIG. 3. When the sensing data related to the water quality or the building condition is provided from the communication terminal 105 of FIG. 1, the controller 310 may temporarily store the sensing data in the storage 330 and then provide the sensing data to the water supply control unit 320 for analysis. In addition, weather information (or data) such as rainfall, precipitation, atmosphere, ozone, fine dust, and temperature provided by public institutions, and other public information (or data) such as traffic, electricity, and gas may be provided to the controller 310, the controller 310 temporarily may store the data provided in the storage 330, and then provide the data provided to the water supply control unit 320.

In addition, the controller 310 may perform various operations at request of the water supply control unit 320, for example, may perform an operation to operate the water pipe scaler 200 disposed inside the water meter 100. To this end, the controller 310 may control the communication interface 300 to communicate with the communication terminal 105 of FIG. 1. Additionally, the controller 310 may control the communication interface 300 to provide the water quality information or the building information to the user terminal 110 or the home pad 115 at the request of the water supply control unit 320. The information may be provided as an online subscription-type paid service, and in the case of a paid service, the controller 310 may also perform an operation for monthly fee settlement. To perform the mentioned operation, the controller 310 may be linked with the water supply control unit 320.

The water supply control unit 320 may monitor the condition of the water pipes through the water supply equipment 100, 105 installed in the water pipe of the water meter 100 in any region or nationwide. In addition, the water supply control unit 320 may also monitor the condition of the building, and the condition of the building may be used for the purpose of disaster prevention response, for the purpose of predicting the correlation between the condition of the building and the condition of the water pipe, and for the purpose of responding in advance. Above all, the water supply control unit 320 may analyze the water quality by analyzing the sensing data provided from the water quality sensor installed inside the water meter 100. As a result of the water quality analysis, in the case that the water quality needs to be improved, the water supply control unit 320 may control the operation of the water pipe scaler 200 such as an ionizer connected to the water pipe. In addition, the water supply control unit 320 may generate control data for a control, provide the control data to the communication terminal 105 of FIG. 1, and allow a control to be performed by the communication terminal 105.

For example, in the case that the water quality has deteriorated by −α, the water supply control unit 320 may control the operation of the water pipe scaler 200 through the control data to compensate by +α to bring the water quality to a normal level. To improve the water quality as mentioned above, sterilization and disinfection may be performed, and the inside of the water pipe may be cleaned. Various measures will not be particularly limited to any one method since it is possible to sufficiently predict what measures the water supply control unit 320 will perform by performing big data collection and analysis. In other words, the water supply control unit 320 may be equipped with an artificial intelligence program to learn the provided data, and may determine action contents through learning, for example, when the learning is made that cleaning the inside of the water pipe is necessary to improve the water quality, the water supply control unit 320 may control cleaning operation to be performed. As mentioned before, since the water pipe scaler 200 according to an embodiment of the present invention improves the water quality by generating static electricity using carbon in tap water, therefore, rust, scale, slime, and general bacteria in the water pipe may also be removed by the operation of the water pipe scaler 200.

Above all, the water supply control unit 320 according to an embodiment of the present invention may predict daily, monthly, and yearly water usage, the water quality changes, the water leakage, and regional water usage by applying the artificial intelligence program. For example, as a result of generating and analyzing big data, in the case that water use patterns for 3 to 5 years is analyzed, the water quality or water usage in a specific period of time to come may be predicted by using the water use patterns. In addition, the changes in the water quality and the water leakage may also be accurately predicted based on the data learned through analysis of aging condition of the water pipe and map learning. In particular, by collecting and analyzing information provided from the Korea Meteorological Administration and Korea Water Resources Corporation regarding the water usage, it may be possible to accurately predict periods of high water usage, such as droughts and days with high temperatures, through the artificial intelligence program. In addition, in the case that some users have experienced the changes in the water quality and the water leakage in the water pipes under a same environment, such as a same material (e.g., copper pipe) and a same year of manufacture, the water supply control unit 320 may predict the changes in the water quality or the water leakage in other users' water pipes through the learning from experience data. In this way, accurate predictions may be made by the artificial intelligence.

In addition, when the water usage of a specific home or region is predicted, the water supply control unit 320 may provide the information based on the prediction result to the user terminal 110 or home pad 115 carried by the users, provide the information to the water supply office device 135 of FIG. 1, and request the information to be used for the water production. The information may be provided to consumers in the form of an online subscription. As the water supply control unit 320 predicts water usage, the energy previously used to overproduce water by about 15% throughout the country and carbon emissions may be reduced.

The storage 330 may temporarily store various data under the control of the controller 310. The storage 330 may temporarily store the sensing data related to the water quality or the building provided from the communication terminal 105 of FIG. 1 and then provide the sensing data to the water supply control unit 320 for the data analysis. Additionally, in the case that the data analysis results are provided in a designated format, the storage 330 may temporarily store the data analysis results and then output the data analysis results to be systematically classified and stored on the web server of FIG. 1.

In addition to the above description, the communication interface 300, the controller 310, the water supply control unit 320, and the storage 330 of FIG. 3 may perform various operations, but since the details are previously disclosed, further description is omitted.

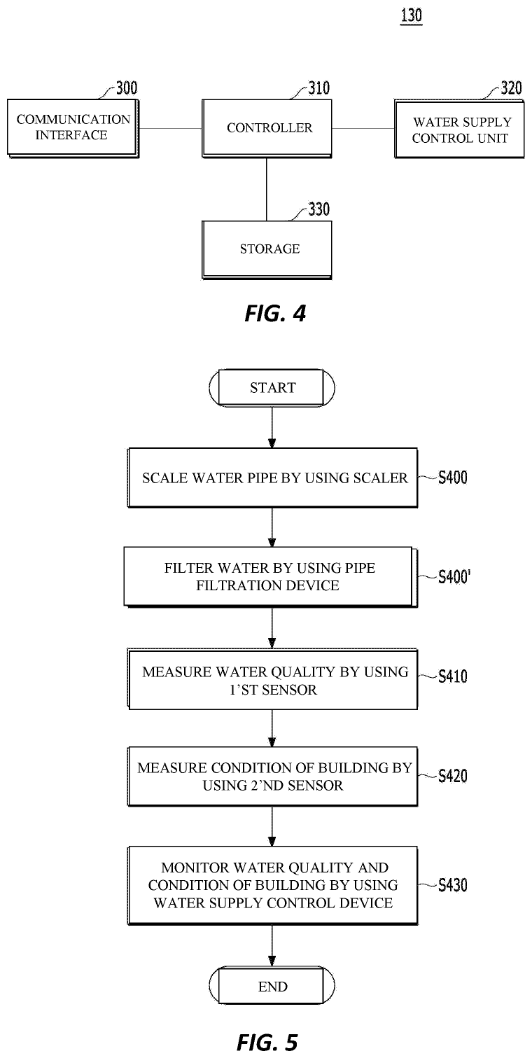

FIG. 4 is a flowchart showing a water supply-related platform service method according to an embodiment of the present invention. Referring to FIG. 4 together with FIG. 1 for convenience of explanation, the water pipe scaler 200 connected to the water pipe of the building according to an embodiment of the present invention may scale the internal condition of the water pipe (S400). The water pipe scaler 200 according to an embodiment of the present invention may operate to improve the water quality by generating static electricity using carbon in tap water. Due to the above configuration, rust, scale, slime, and general bacteria in the water pipe may be removed by installing the water pipe scaler 200. The water pipe scaler 200 may be remotely controlled by the water supply control device 130 of FIG. 1.

Additionally, the first sensor, such as the water quality sensor, installed in the water pipe, may measure the water quality in the water pipe (S410). The mercury, conductivity, turbidity, TDS, salinity, ORP, and etc. may be measured by using the water quality measurement. Since various types of sensors may be used to measure the relevant characteristics, embodiments of the present invention will not be specifically limited to any one sensor type.

The second sensor, such as the vibration sensor or the ground sensor, installed in the building or the ground, may measure the condition of the building or the ground (S420). The building safety information generated by measuring the condition of the building by the second sensor may be used for the disaster prevention response purposes, and, by using the correlation between the building vibration and the water pipes, may be used to predict the condition changes by the correlation of the building and the water pipes and take appropriate responses according to the prediction results.

The water supply control device 130 may monitor the water quality and the ground condition of the building based on the sensing data from the first sensor and the second sensor (S430). Through monitoring, the water quality information and the building condition information may be provided to the user terminal 110 or home pad 115 of the building owner or the residents living in the building. The various information provided to the building owner or the residents may be provided in a subscription form.

In addition to the above description, the water supply equipment 100, 105, the home pad 115, the user terminal 110, the communication network 120, the water supply control device 130, the water supply office device 135, and the administrator device 140 of FIG. 1 according to an embodiment of the present invention may perform various operations, but since the details are previously disclosed, further description is omitted.

Meanwhile, although the description that all the components constituting the embodiment of the present invention are combined or operated in combination has been described, the present invention is not limited to the embodiments. That is, if the components fall within spirit of the present invention, all components may operate by selectively combining one or more. In addition, all components may be implemented as independent hardware, and some or all of the components may be selectively combined to be implemented as a computer program that includes a program module that performs some or all of the functions in one or more pieces of hardware. Codes and code segments constituting the computer program may be easily deduced by a person skilled in the art of the present invention. Such computer programs may be stored in non-transitory computer readable media, and the embodiments of the present invention may be implemented by being read and executed by a computer.

The non-transitory computer readable media may include a medium that stores data semi-permanently and can be read by a device, rather than a medium that stores data for a short period of time, such as a register, cache, or memory. Specifically, the above-described programs may be stored and provided on the non-transitory computer readable media such as compact disc (CD), digital versatile disc (DVD), hard disk, Blu-ray disk, universal serial bus (USB), memory card, and read only memory (ROM).

In the description, although the disclosure that all the components constituting the embodiment of the present invention are combined or operated in combination has been described, the present invention is not limited to embodiments. That is, if the components fall within the spirit of the present invention, all components may operate by selectively combining one or more. In addition, terms such as “include,” “comprise,” or “have” used in the description may mean that the corresponding component may be included, unless clearly stated to the contrary, and should be interpreted as being able to include other components rather than to exclude other components. All terms including technical or scientific terms, unless otherwise defined, have same meaning as generally understood by a person of ordinary skill in the technical field to which the present invention pertains. Commonly used terms, such as terms defined in a dictionary, should be interpreted as consistent with a contextual meaning of the related technology, and should not be interpreted in an idealized or overly formal sense unless explicitly defined in the present invention.

The above-mentioned description is merely an illustrative explanation of the technical idea of the present invention, and various modifications and variations may be made by those skilled in the art without departing from the essential characteristics of the present invention. Accordingly, the embodiments disclosed in the present invention are not intended to limit the technical idea of the present invention, and are disclosed for illustrative purposes, and the scope of the technical idea of the present invention is not limited by the embodiments. The scope of protection of the present invention should be interpreted in accordance with the claims below, and all technical ideas within the equivalent scope should be construed as being included in the scope of rights of the present invention.

The Abstract at the end of this disclosure is provided to comply with 37 C.F.R. § 1.72 (b) to permit the reader to quickly ascertain the nature of the technical disclosure. It is submitted with the understanding that it will not be used to interpret or limit the scope or meaning of the claims.

What is claimed is:

1. A water purification system for a smart building comprising:
a water pipe scaler connected to a water pipe of a building and to scale an internal condition of the water pipes;
a water pipe filtration device to filter a water supplied to the water pipe;
a first sensor for measuring a water quality of the water flowing in the water pipe;
a second sensor installed at the building or a ground of the building to measure a condition of the building or the ground; and
a water supply control device to monitor the water quality and the condition of the building or the ground based on sensing data of the first sensor and the second sensor.

2. The water purification system of claim 1 wherein the water pipe filtration device is:
disposed in a bypass pipe connected to the water pipe; and
selectively opened and closed.

3. The water purification system of claim 1 wherein the water pipe filtration device is:
composed of a plurality of devices;
disposed in each bypass pipe connected to the water pipe; and
selectively opened and closed.

4. The water purification system of claim 1 wherein the water supply control device operates the water pipe scaler and the water pipe filtration device based on an analysis result of first sensing data of the first sensor.

5. The water purification system of claim 1 wherein the water supply control device:
comprises a vibration sensor and a ground subsidence sensor as the second sensor;
collects and analyzes second sensing data of the second sensor; and
performs a disaster prevention response operation based on an analysis result of the second sensing data.

6. The water purification system of claim 1 wherein the water supply control device notifies a condition of the water quality and the condition of the building or the ground to a user terminal owned by an owner of the building or a resident living in the building.

7. The water purification system of claim 1, wherein the water supply control device:
uses an artificial intelligence (AI) program to analyze first sensing data of the first sensor and public data acquired from public institutions related to the water quality;
determines a water usage in a random area based on a result of the analysis; and
ensures a water production according to a result of the determination.
